(19) United States
(12) Patent Application Publication
(10) Pub. No.: US 2016/0213658 A1
(43) Pub. Date: Jul. 28, 2016

(54) PHARMACEUTICAL COMPOSITIONS OF ROFLUMILAST AND PROCESS FOR PREPARATION THEREOF

(71) Applicant: HETERO RESEARCH FOUNDATION, Hyderabad, Andhra Pradesh (IN)

(72) Inventors: BANDI PARTHASARADHI REDDY, HYDERABAD, ANDHRA PRADESH (IN); PODILI KHADGAPATHI, HYDERABAD, ANDHRA PRADESH (IN); POTHIREDDY VENKATESWAR REDDY, HYDERABAD, ANDHRA PRADESH (IN); MUPPIDI VANAJA KUMARI, HYDERABAD, ANDHRA PRADESH (IN)

(73) Assignee: Hetero Research Foundation

(21) Appl. No.: 14/917,709

(22) PCT Filed: Sep. 11, 2014
(86) PCT No.: PCT/IN2014/000588

§ 371 Date: Mar. 9, 2016

(30) Foreign Application Priority Data
Sep. 13, 2013 (IN) 4119/CHE/2013

(51) Int. Cl. A61K 31/44; A61K 9/20

(52) U.S. Cl. CPC A61K 31/44; A61K 9/2054; A61K 9/2018

(57) ABSTRACT

The present invention relates to pharmaceutical compositions of roflumilast. More particularly, the present invention relates to pharmaceutical tablet compositions of roflumilast and process for preparing the same.

### PRIORITY

This patent application claims priority to Indian patent application number 4119/CHE/2013, filed on Sep. 13, 2013, the contents of which are incorporated by reference herein in their entirety.

### FIELD OF THE DISCLOSURE

The present disclosure relates to pharmaceutical compositions comprising Roflumilast or a pharmaceutically acceptable salt thereof.

### BACKGROUND

Roflumilast is chemically described as N-(3,5-dichloropyridin-4-yl)-3-cyclo propylmethoxy-difluoromethoxy-benzamide. It's structural formula as follows:

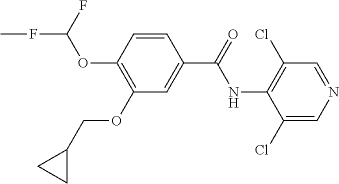

Roflumilast is commercially available from FOREST Pharmaceuticals as DALIRESP® as oral tablets containing equivalent to 500 mcg of Roflumilast. DALIRESP® is indicated for reducing the risk of chronic obstructive pulmonary disease exacerbations in patients with severe chronic obstructive pulmonary disease associated with chronic bronchitis, comprising administering to the patient.

U.S. Pat. No. 5,712,298 assigned to BYK disclose roflumilast.

U.S. Pat. No. 8,431,154 disclose roflumilast tablet or pellet composition prepared by aqueous granulation using solution of polyvinylpyrrolidone.

There is a need to develop alternative compositions of Roflumilast using simplified process. Accordingly, inventors of the present invention have developed novel compositions of Roflumilast and process for preparing the same.

### SUMMARY

The present invention relates, to pharmaceutical compositions of roflumilast and one or more pharmaceutically acceptable excipients and process for preparation thereof.

One embodiment of the present invention relates to pharmaceutical tablet composition comprising roflumilast and one or more pharmaceutically acceptable excipients; wherein the composition is prepared by non-aqueous granulation process.

Another embodiment of the present invention relates to pharmaceutical composition comprising roflumilast and one or more pharmaceutically acceptable excipients; wherein said roflumilast is present in an amount of 0.2% to 0.4% by weight based on total weight of the composition.

Another embodiment of the present invention relates to pharmaceutical tablet composition comprising roflumilast in an amount of 0.2% to 0.4% by weight based on total weight of the composition, microcrystalline cellulose, mannitol and at least one other pharmaceutically acceptable excipient; wherein the composition is prepared by non-aqueous granulation process.

Other embodiment of the present invention relates to process of preparing a pharmaceutical tablet of roflumilast comprising the following steps: (a) blending the excipients to form a dry mixture; (b) granulating the dry mixture of step (a) using solution of roflumilast with mixture of isopropyl alcohol and dichloromethane, followed by drying and milling to get the desired size granules; (c) optionally blending the granules of step (b) with one or more excipients; (d) lubricating the granules of step (b) or blend of step (c) and finally; (e) compressing the lubricated blend of step (d) in to tablets.

Also included in the present invention is the use of roflumilast composition for reducing the risk of chronic obstructive pulmonary disease exacerbations in patients with severe chronic obstructive pulmonary disease associated with chronic bronchitis, comprising administering to the patient.

### DETAILED DESCRIPTION

The term “active ingredient” or “active agent” or “drug” used interchangeably, is defined to mean active drug (e.g. roflumilast), that induce a desired pharmacological or physiological effect.

The term “pharmaceutically acceptable” as used herein means that which is useful in preparing a pharmaceutical composition that is generally safe and non-toxic.

The term “excipients” as used herein means a component of a pharmaceutical product that is not an active ingredient such as, for example, fillers, diluents, carriers and the like. The excipients that are useful in preparing a pharmaceutical composition are generally safe and non-toxic.

By the term “solid dosage form” or “dosage form” or “composition” as used herein refers to a solid dosage form suitable for administration, such as a tablet, capsule, mini-tablets, granules, and the like.

As used in the specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

As used in this specification and the appended claims, the singular forms “a”, “an”, and “the” include plural references unless the context clearly dictates otherwise. Thus for example, a reference to “a method” or “a process” includes one or more methods, one or more processes and/or steps of the type described herein and/or which will become apparent to those persons skilled in the art upon reading this disclosure and so forth.

One embodiment of the present invention relates to pharmaceutical composition comprising roflumilast and one or more pharmaceutical acceptable excipients; wherein said roflumilast comprise in an amount of 0.2% to 0.4% by weight based on total weight of the composition.

Pharmaceutical compositions of roflumilast according to the present invention further comprise one or more excipients selected from diluents, disintegrants, binders, glidants and lubricants.

Suitable diluents according to the present invention include one or more of mannitol, microcrystalline cellulose, lactose, starch, dicalcium phosphate, sucrose, sorbitol and calcium carbonate and the like.

Particularly diluents of the present invention includes one or combination of mannitol, microcrystalline cellulose and lactose.

Suitable disintegrants include, by way of example and without limitation starches such as maize starch, potato starch, pre-gelatinized and modified starches, microcrystalline cellulose, hydroxypropyl methylcellulose, hydroxypropyl cellulose, polacrillin potassium, croscarmellose sodium, sodium starch glycolate, carboxymethyl cellulose calcium and the like or combinations thereof.

Suitable binders include, by way of example and without limitation maize starch, pregelatinized starch, hydroxypropyl cellulose, hydroxypropyl methylcellulose, powdered acacia, gelatin, guar gum, carbomers and the like, and combinations thereof.

Suitable lubricants include, by way of example and without limitation, magnesium stearate, calcium stearate, zinc stearate, mineral oil, stearic acid, fumaric acid, palmitic acid, talc, carnauba wax, hydrogenated vegetable oils, mineral oil, polyethylene glycols, sodium stearyl fumarate and the like or combinations thereof.

Suitable glidants include, by way of example and without limitation, colloidal silicon dioxide, magnesium silicate, magnesium trisilicate, talc, and other forms of silicon dioxide, such as aggregated silicates and hydrated silica and the like, and combinations thereof.

Another embodiment of the present invention relates to pharmaceutical tablet composition comprising roflumilast and one or more pharmaceutically acceptable excipients; wherein the composition is prepared by non-aqueous granulation process.

Non-aqueous granulation according to the present invention comprise the steps of: (a) blending the excipients to form a dry mixture; (b) granulating the dry mixture of step (a) using solution of roflumilast using a non-aqueous solvent or a mixture of non-aqueous solvents.

Non-aqueous granulation according to the present invention was carried out using organic solvents selected from one or more of isopropyl alcohol, dichloromethane, ethanol, methanol and mixtures thereof.

Another embodiment of the present invention relates to pharmaceutical tablet composition comprising roflumilast in an amount of 0.2% to 0.4% by weight based on total weight of the composition, microcrystalline cellulose, mannitol and at least one other pharmaceutically acceptable excipient; wherein the composition is prepared by non-aqueous granulation process.

Another embodiment of the present invention relates to the process of preparing tablet compositions of roflumilast according to the present invention involves the steps of: (a) blending the excipients to form a dry mixture; (b) granulating the dry mixture of step (a) using solution of roflumilast with mixture of isopropyl alcohol and dichloromethane, followed by drying and milling to get the desired size granules; (c) optionally blending the granules of step (b) with one or more excipients; (d) lubricating the granules of step (b) or blend of step (c) and finally; (e) compressing the lubricated blend of step (d) in to tablets.

According to the present invention, the usage of roflumilast with mixture of isopropyl alcohol and dichloromethane as a granulating medium resulted in a better dissolution profile of the tablet compositions prepared.

In an another aspect, the present invention relates to pharmaceutical tablet composition comprising roflumilast, 10% to 20% by weight of microcrystalline cellulose, and 75% to 90% by weight of mannitol based on total weight of the composition; wherein the composition is prepared by non-aqueous granulation process.

The compositions according to the present invention are devoid of povidone in its entirety.

The tablets of the present invention may optionally be coated with an aqueous or non aqueous solution or dispersion of film forming agents. If desired, the film coat may be an aqueous moisture barrier. The coating solution mainly comprises of film forming polymers and one or more of plasticizers, opacifier, surfactant, anti tacking agents, coloring agent and the like.

The coating according to the present invention is applied by solubilising or suspending the excipients in solvents such as isopropyl alcohol, water, acetone, ethanol, methylene chloride and the like or mixtures thereof.

Another object of the present invention is to provide improved content uniformity of roflumilast tablets despite of its lower dose in the composition.

Content uniformity of the tablets was determined using 10 random tablets, by performing an HPLC assay to measure the amount of active ingredient in each tablet, and comparing the amount of active ingredient in each tablet to the labeled amount of active ingredient. The standard deviation and relative standard deviation were determined accordingly.

Content uniformity of the tablets tested ranged from 95.8% to 98.2%. RSD (relative standard deviation, expressed as a percentage of the mean) was found to be lower than 1.0% indicating that the uniformity of tablets was high.

Pharmaceutical compositions of the present invention comprising therapeutically effective amount of roflumilast are useful for reducing the risk of chronic obstructive pulmonary disease exacerbations in patients with severe chronic obstructive pulmonary disease associated with chronic bronchitis.

### EXAMPLES

The following examples further describe and demonstrate particular embodiments within the scope of the present invention. The examples are given solely for illustration and are not to be construed as limitations as many variations are possible without departing from spirit and scope of the invention. It is obvious to those skilled in the art to find out the composition for other dosage forms and substitute the equivalent excipients as described in this specification or with the one known to the industry.

### Example 1

### Tablet Compositions of Roflumilast

| Ingredients | mg/tablet |
|---|---|
| Dry mix: | |
| Mannitol | 118.50 |
| Microcrystalline cellulose | 18.00 |
| Binder solution: | |
| Roflumilast | 0.50 |
| Isopropyl alcohol | q.s. |
| Dichloromethane | q.s. |
| Lubrication: | |
| Magnesium stearate | 3.00 |
| Core tablet weight | 140.00 |
| Film coating: | |
| Opadry ® yellow | 2.80 |
| Purified water | q.s. |
| Total tablet weight | 142.80 |

Preparation Method:
1. Microcrystalline cellulose, mannitol were sifted together through mesh #40, loaded into rapid mixer granulator and blended for 10 minutes,
2. drug solution was prepared using roflumilast, isopropyl alcohol and dichloromethane,
3. blend of step 1 was granulated using drug solution of step 2 followed by drying and sifting to get the desired granules,
4. extragranular magnesium stearate was sifted through mesh #60 sieve,
5. dried granules of step 3, were lubricated with magnesium stearate of step 4,
6. lubricated blend of step 5 was compressed into tablets using suitable punches,
7. tablets of step 6 were film coated using Opadry® yellow dispersion.

### Example 2

### Compositions of Roflumilast

| Ingredients | mg/tablet |
|---|---|
| Dry mix: | |
| Mannitol | 118.50 |
| Microcrystalline cellulose | 20.00 |
| Binder solution: | |
| Roflumilast | 0.50 |
| Isopropyl alcohol | q.s. |
| Dichloromethane | q.s. |
| Extragranular: | |
| Maize starch | 18.00 |
| Lubrication: | |
| Magnesium stearate | 3.00 |
| Core tablet weight | 160.00 |
| Film coating: | |
| Opadry ® yellow | 2.80 |
| Purified water | q.s. |
| Total tablet weight | 162.80 |

### Preparation Method:

1. Microcrystalline cellulose, mannitol were sifted together through mesh #40, loaded into rapid mixer granulator and blended for 10 minutes,
2. drug solution was prepared using roflumilast, isopropyl alcohol and dichloromethane,
3. blend of step 1 was granulated using drug solution of step 2 followed by drying and sifting to get the desired granules,
4. extragranular maize starch was sifted through mesh #40, dried granules of step 3 were blended with maize starch of step 4,
5. extragranular magnesium stearate was sifted through mesh #60 sieve,
6. blended granules of step 5, were lubricated with magnesium stearate of step 6,
7. lubricated blend of step 7 was compressed into tablets using suitable punches or filled into capsules,
8. tablets of step 8 were film coated using Opadry® yellow dispersion.

### Example 3

### Tablet Compositions of Roflumilast

| Ingredients | mg/tablet |
|---|---|
| Dry mix: | |
| Mannitol | 68.25 |
| Macrocrystalline cellulose | 8.00 |
| Binder solution: | |
| Roflumilast | 0.50 |
| Isopropyl alcohol | q.s. |
| Dichloromethane | q.s. |
| Lubrication: | |
| Magnesium stearate | 3.00 |
| Colloidal silicon dioxide | 0.25 |
| Total tablet weight | 80.00 |

### Preparation Method:

1. Microcrystalline cellulose, mannitol were sifted together through mesh #40, loaded into rapid mixer granulator and blended for 10 minutes,
2. drug solution was prepared using roflumilast, isopropyl alcohol and dichloromethane,
3. blend of step 1 was granulated using drug solution of step 2 followed by drying and sifting to get the desired granules,
4. extragranular colloidal silicon dioxide was sifted through mesh #40 sieve,
5. extragranular magnesium stearate was sifted through mesh #60 sieve,
6. dried granules of step 3, were blended with colloidal silicon dioxide of step 4,
7. blend of step 6, was lubricated with magnesium stearate of step 5,
8. lubricated blend of step 7 was compressed into tablets using suitable punches.

### Dissolution Data:

Dissolution test was performed for tablets prepared as per Example 3 and Daliresp 500 mcg tablets using USP apparatus II, at 50 rpm in 1000 ml of 6.8 Phosphate buffer containing 0.1% SLS.

Dissolution (%)

| | Time in minutes | | | | | |
|---|---|---|---|---|---|---|
| | 10 min | 15 min | 20 min | 30 min | 45 min | 60 min |
| Example-3 | 76 | 90 | 93 | 95 | 97 | 98 |
| Daliresp | 28 | 57 | 82 | 93 | 100 | 101 |
| 500 mcg tablets | | | | | | |

From the above table, it was observed that tablet composition of present invention having comparable dissolution profile compared with Daliresp 500 mcg tablets.

### Content Uniformity Test Data:

The tablet compositions prepared according to the present invention were subjected to content uniformity test.

| | |
|---|---|
| Content | Assay |

| Uniformity | mcg | % label claim |
|---|---|---|
| CU-1 | 481 | 96.2 |
| CU-2 | 490 | 98.0 |
| CU-3 | 489 | 97.8 |
| CU-4 | 490 | 98.0 |
| CU-5 | 486 | 97.2 |
| CU-6 | 479 | 95.8 |
| CU-7 | 487 | 97.4 |
| CU-8 | 480 | 96.0 |
| CU-9 | 491 | 98.2 |
| CU-10 | 485 | 97.0 |
| Mean | 486 | 97.2 |
| SD | 0.0 | 0.9 |
| RSD | 0.00 | 0.93 |
| Minimum | 480 | 95.8 |
| Maximum | 490 | 98.2 |

From the above table, RSD was found to be lower than 1.0% indicating that the uniformity of tablets prepared was high.

1. A pharmaceutical tablet composition comprising roflumilast and one or more pharmaceutically acceptable excipients; wherein the composition is prepared by anon-aqueous granulation process.

2. The pharmaceutical tablet composition of claim 1, wherein the non-aqueous solvent is isopropyl alcohol, dichloromethane, ethanol, methanol, or a combination thereof.

3. The pharmaceutical tablet composition of claim 1, wherein said non-aqueous granulation process comprise the steps of: (a) blending the one or more pharmaceutically acceptable excipients to form a dry mixture; and (b) granulating the dry mixture of step (a) using a solution of roflumilast with anon-aqueous solvent or a mixture of non-aqueous solvents.

4. A pharmaceutical composition comprising roflumilast and one or more pharmaceutical acceptable excipients; wherein said roflumilast is present in an amount of 0.2% to 0.4% by weight, based on total weight of the composition.

5. The pharmaceutical composition according to claim 4, wherein said pharmaceutically acceptable excipient is a diluent, a binder, a disintegrant, a glidant, a lubricant, or a combination thereof.

6. The pharmaceutical composition of claim 4, in the form of granules, a tablet or a capsule.

7. The pharmaceutical composition of claim 4 in the form of a tablet comprising, microcrystalline cellulose, mannitol and at least one other pharmaceutically acceptable excipient; wherein the composition is prepared by a non-aqueous granulation process.

8. The process of preparing a pharmaceutical tablet compositions of roflumilast comprising the steps of: (a) blending microcrystalline cellulose, mannitol, and at least one other pharmaceutical acceptable excipient to form a dry mixture; (b) granulating the dry mixture of step (a) using a solution of roflumilast with a mixture of isopropyl alcohol and dichloromethane, followed by drying and milling to provide granules; (c) optionally blending the granules of step (b) with one or more pharmaceutically acceptable excipients; (d) lubricating the granules of step (b) or blend of step (c) and; (e) compressing the lubricated blend of step (d) in to tablets.

9. The pharmaceutical composition of claim 1, wherein the composition is devoid of povidone.

10. (canceled)

11. The pharmaceutical composition of claim 4, wherein the composition is devoid of povidone.

12. The method of claim 8, wherein the composition is devoid of povidone.
